(19) United States
(12) Patent Application Publication
(10) Pub. No.: US 2006/0206031 A1
(43) Pub. Date: Sep. 14, 2006

(54) Blood pressure detecting device, blood pressure detecting method, blood pressure detecting program, and strain sensor for blood pressure detection

(76) Inventor: Motoharu Hasegawa, Tokyo (JP)

Correspondence Address: RADER FISHMAN & GRAUER PLLC, LION BUILDING, 1233 20TH STREET N.W., SUITE 501, WASHINGTON, DC, 20036, US

(21) Appl. No.: 11/370,020

(22) Filed: Mar. 8, 2006

(30) Foreign Application Priority Data
Mar. 9, 2005 (JP) 2005-66237
Mar. 31, 2005 (JP) 2005-102048

(51) Int. Cl. A61B 5/02

(52) U.S. Cl. 600/490; 600/485; 600/500

(57) ABSTRACT

The present invention may detect a maximum blood pressure and a minimum blood pressure from a viewpoint different from that of a conventional blood pressure measuring method. The present invention propose a strain sensor for blood pressure detection, comprising: a pressure transducer including: a metal thin plate for receiving a beat of a living body; and a strain gauge provided on a surface of the metal thin plate, for detecting a pressure based on the beat propagating through the metal thin plate.

### BACKGROUND OF THE INVENTION

1. Field of the Invention

The present invention relates to a blood pressure detecting device and a blood pressure detecting method, each of which is capable of detecting both a maximum blood pressure and a minimum blood pressure (maximum and minimum blood pressures) of a living body based on a pulse wave propagating through an artery of the living body, a blood pressure detecting program executed by a computer based on the blood pressure detecting method, and a strain sensor for blood pressure detection that can be used for the blood pressure detecting device and the blood pressure detecting method.

2. Description of the Related Art

A noninvasive blood pressure measuring method may include an auscultatory method, an oscillometric method, and a tonometry method. The auscultatory method is a method of listening to a Korotkoff sound with a stethoscope. The Korotkoff sound appears and disappears in the process in which a blood vessel is opened to start blood flow after a state in which the blood vessel is compressed to stop the blood flow. To be more specific, a cuff is wound around an upper arm and air is injected into the cuff to compress the blood vessel. At this time, the arm is compressed at a cuff pressure that exceeds a maximum blood pressure to completely occlude a brachial artery, thereby blocking the blood flow to the downstream side. After that, the air is gradually removed from the cuff to decrease the compression pressure (cuff pressure) applied to the upper arm by the cuff. When the cuff pressure becomes lower than the maximum blood pressure, the blood flow starts again. Then, intermittent blood flow occurs in accordance with the beat. A sound that appears at each time is the Korotkoff sound. When the cuff pressure further decreases and becomes lower than a minimum blood pressure, the brachial artery is fully opened. Therefore, steady flow occurs, so that the Korotkoff sound disappears. When the Korotkoff sound is listened to with the stethoscope placed on a peripheral side of a region to which the cuff is attached and above a brachial artery beat region, a pressure at a time when the Korotkoff sound appears is determined as the maximum blood pressure and a pressure at a time when the Korotkoff sound disappears is determined as the minimum blood pressure.

As in the auscultatory method, according to the oscillometric method, the blood flow is stopped and started using the cuff. This measurement method is based on an oscillation phenomenon of an internal cuff pressure that is caused by the beat of an artery at a time when the artery is compressed by the cuff. When the brachial artery is occluded by the cuff and then the cuff pressure gradually decreases, there are a point when an amplitude of the cuff pressure significantly increases and a point when the amplitude thereof becomes significantly small. Therefore, a pressure at the point when the amplitude significantly increases is determined as the maximum blood pressure and a pressure at the point when the amplitude becomes significantly small is determined as the minimum blood pressure.

The tonometry method is a method of directly detecting a pressure of an artery using a pressure sensor. To be specific, a surface of a living body is pressed with a flat plate to flatly deform an artery. At this time, a pressure at which the artery is maintained in a flat state is detected by the pressure sensor and converted into an electrical signal to obtain a pulse waveform. The maximum blood pressure and the minimum blood pressure are determined from a maximum point of the obtained pulse waveform and a minimum point thereof based on a relationship between a press pressure and a blood pressure value, which are obtained in advance. For example, a blood pressure measuring device using the oscillometric method is described in JP 05-038332 A and a blood pressure measuring device using the tonometry method is described in JP 10-243929 A.

However, each of the above-mentioned blood pressure measurement methods has problems. Therefore, blood pressure detection cannot be performed with accuracy.

According to the auscultatory method, points when the Korotkoff sound appears and disappears are normally determined by the ears of a human. Therefore, it is likely to cause an error depending on a person who executes the measurement. In addition, skill is required. Even when a transducer such as a microphone is used instead of the ears of the human, there is a problem in which it is likely to include a noise.

In addition, according to the auscultatory method, the Korotkoff sound follows the process of typical sound quality change in an ideal state, so that the points when the sound appears and disappears can be substantially accurately picked up. However, this method has disadvantages in that a preferable Korotkoff sound does not necessarily appear and the Korotkoff sound depends on the personal property of a person to be examined and a measurement condition. That is, the Korotkoff sound depends on the personal property of the person to be examined, such as a size of an arm, a blood pressure value, a strong heart or a weak heart, the occurrence or absence of arrhythmia, the occurrence or absence of heart failure, or the occurrence or absence of an abnormal reduction in blood pressure and the measurement condition such as a press pressure of the stethoscope. Therefore, the points when the Korotkoff sound appears and disappears cannot be determined in some cases. Thus, as long as the Korotkoff sound is used, even when exact frequency analysis is to be performed to determine the points by a program, a specific Korotkoff sound frequency distribution band depends on the individual, so it is difficult to accurately measure the blood pressure of all persons.

FIG. 9 shows the Korotkoff sound that is converted into an oscillation waveform. This oscillation waveform is divided into a SwanI point, a SwanI point, a SwanII point, a SwanIV point, and a SwanV point based on the frequency. The maximum blood pressure corresponds to the SwanI point. The minimum (lowest) blood pressure corresponds to the SwanIV point or the SwanV point. The SwanIV point is normally determined as the minimum blood pressure. As shown in FIG. 9, it is preferable to clear the SwanI point and the SwanIV point. However, both points become unclear depending on the personal property of a person to be examined and a measurement condition in some cases.

Even to this day, there is a discussion as to whether the minimum blood pressure corresponds to the SwanIV point at which the sound becomes weaker or the SwanV point at which the sound disappears. Although the SwanIV point is determined as the minimum blood pressure in acceptable convention, a relationship between the Korotkoff sound and the minimum blood pressure is unclear. This reason is as follows. That is, when the blood vessel that is being occluded by the cuff pressure is fully opened at the minimum blood pressure, blood rushes therethrough. However, at this time, an instant blood flow quantity and an instant blood flow velocity become larger. Therefore, an arterial lumen wall oscillates, so that a pseudo Korotkoff sound appears at a time when the cuff pressure is lower than the minimum blood pressure in some cases. Even when the Korotkoff sound is converted into the waveform as described above, there is a case where it is difficult to determine the minimum blood pressure. On the other hand, even when the brachial artery is completely occluded at the cuff pressure that is equal to or larger than the maximum blood pressure, pulsation flow from a central side collides with a central end portion of the occluded artery, so that the pseudo Korotkoff sound appears in some cases. If this is analyzed by conventional frequency analysis and the determination is made, it is likely to display an erroneous maximum blood pressure.

According to the oscillometric method, the maximum blood pressure and the minimum blood pressure are determined based on the oscillation frequency. However, processing to be performed for a misleading case is not fixed, so that a target point set to display the maximum blood pressure and the minimum blood pressure is unclear. That is, although the oscillation of the internal cuff pressure is processed by a computer using a predetermined program, there is no program that can be used for all cases, so accurate blood pressures cannot be detected depending on cases.

In contrast to this, the tonometry method has an advantage in that a pressure waveform is obtained for each heart beat. However, when the blood pressure is to be accurately measured, a measurement device that is sophisticated is necessary and thus expensive. There are many limits at the time of measurement, so that simple measurement cannot be performed. For example, in the tonometry method, the blood vessel is flatly pressed to balance the blood vessel and the pressure sensor. Therefore, it is necessary to use a special pressure sensor including press pressure providing means capable of injecting a fluid or air into the pressure sensor to press the surface of the living body from an inner portion thereof and press pressure controlling means for controlling a press pressure to the surface thereof. In addition to this, in the tonometry method, a value of the pulse wave resulting from the beat is directly determined as a blood pressure value, so it is necessary to accurately detect the pressure of the artery. Therefore, a pressure sensor having a size smaller than that of the blood vessel is required. Further, a position of a blood vessel located in the inner portion of the living body cannot be grasped, so it is necessary to set a large number of pressure sensors in advance and select a pressure sensor that detects a most suitable pressure of the blood vessel. In order to meet those needs, a very small and expensive pressure sensor and an advanced technique for finely arranging the pressure sensors are required, so that a resultant device must be expensive.

Even in the case of the device obtained as described above, there is a disadvantage in that it is hard to detect an accurate blood pressure. This reason is as follows. That is, there are many measurement limits such as the need to suitably press the blood vessel to flatten and the need to maintain a balance with an internal pressure of the blood vessel within a region in which the blood vessel exists. Therefore, even when the person to be examined slightly moves during the measurement, this slight movement causes a noise, with the result that accurate measurement cannot be performed.

As described above, although the auscultatory method, the oscillometric method, and the tonometry method have various disadvantages, these measurement methods are actually used in a range in which the disadvantages may be acceptable at a blood pressure measurement location. However, even if the disadvantages are eliminated, the measurement methods cannot be actually employed in some cases. For example, in the oscillometric method, an abnormal low blood pressure cannot be measured. To be more specific, for example, when a blood pressure becomes the abnormal low blood pressure equal to or lower than 50 mmHg by shock or the like and thus a cardiac output is low, the blood pressure cannot be measured. Therefore, no oscillometric method is employed to measure the blood pressure of a severe patient in an operation room or an intensive-care unit (ICU). The oscillometric method cannot be employed for a special blood pressure test, for example, blood pressure measurement during exercise stress, such as a cardiovascular exercise stress test. This is because, various vibrations occur, so that amplitude processing performed by a computer cannot be performed.

Even in the auscultatory method, when the surroundings are noisy or when the heart beat is weak, the measurement is difficult. When the heart beat weakens, the Korotkoff sound becomes weaker. When the body is moved by a treadmill or an ergometer, noise results from the vibration of bones or the movement of muscles. Therefore, it is difficult to determine the Korotkoff sound in any of the cases. Even in the tonometry method, the measurement cannot be performed unless a rest state is set. Therefore, it is unlikely to perform the measurement during an exercise.

### SUMMARY OF THE INVENTION

Thus, the present invention may detect a maximum blood pressure and a minimum blood pressure from a viewpoint different from that of a conventional blood pressure measuring method. In other words, the present invention may provide a blood pressure detecting device capable of detecting the maximum blood pressure and the minimum blood pressure from another viewpoint without blood pressure detection based on Korotkoff sounds, thereby obtaining more accurate blood pressure values.

Furthermore, the present invention may obtain a blood pressure detecting device capable of detecting the maximum blood pressure and the minimum blood pressure even when a heart rate reduces.

In addition, the present invention may obtain a blood pressure detecting device capable of detecting the maximum blood pressure and the minimum blood pressure even when a body moves during an exercise or the like.

The present invention may obtain a strain sensor for blood pressure detection, a blood pressure detecting method, and a blood pressure detecting program, which are used for the blood pressure detecting device. In order to achieve the above advantages, the present invention may provide a strain sensor for blood pressure detection, including: a pressure transducer including a metal thin plate for receiving a beat of a living body; and a strain gauge provided on a surface of the metal thin plate, for detecting a pressure based on the beat propagating through the metal thin plate.

The pressure transducer included in the strain sensor for blood pressure detection may include the metal thin plate that is in contact with the living body to receive the beat of the living body. Therefore, the strain gauge provided on a rear surface of the metal thin plate can be prevented from exposing to an outside, thereby protecting the strain gauge. The strain gauge can be prevented from bending by, for example, a finger that is in contact therewith. The pressure transducer further may include the strain gauge provided on one surface of the metal thin plate, for detecting the pressure based on the beat propagating through the metal thin plate. Therefore, a pressure of a blood vessel can be detected as strain of the strain gauge through the metal thin plate.

Because the strain sensor for blood pressure detection may include the pressure transducer, the beat of the living body can be picked up as a pressure signal and can be used for accurate blood pressure detection.

The metal thin plate that can be used for the pressure transducer has a diameter of 5 mm to 20 mm and a thickness that is equal to or smaller than 2 mm and corresponds to a thickness capable of maintaining a thin plate shape. The metal thin plate can be made of a copper alloy. Because the metal thin plate has the diameter of 5 mm to 20 mm and the thickness that is equal to or smaller than 2 mm and corresponds to the thickness capable of maintaining the thin plate shape and made of the copper alloy, the beat of the blood vessel can be accurately picked up. That is, because the diameter of the metal thin plate is set to 5 mm to 20 mm, it is not easily displaced from a position immediately above a beat region from which a pulse wave is detected and the pulse wave is hardly affected by a noise. In addition, because the metal thin plate has the thickness that is equal to or smaller than 2 mm and corresponds to the thickness capable of maintaining the thin plate shape, it is possible to sufficiently transfer the beat from the blood vessel to the pressure transducer. Because the copper alloy is used as a material of the metal thin plate, the pressure applied to the surface of the metal thin plate can be sufficiently transferred to the pressure transducer. The metal thin plate has excellent restitution force in a case where it is strained. Therefore, a preferable sensitive strain sensor for blood pressure detection is obtained. When a phosphor bronze plate that is the copper alloy plate is used, it is hardly affected by heat, so that the beat can be accurately transferred to the pressure transducer.

According to the strain sensor for blood pressure detection in which a semiconductor strain gauge can be used as the strain gauge, because the semiconductor strain gauge has a high gauge factor and is a small size, it is possible to obtain a sensitive and compact strain sensor for blood pressure detection. Therefore, even when the strain sensor is attached to the body for an exercise, an obstruction does not occur. In addition, the strain sensor is hardly affected by a noise resulting from the exercise. The strain sensor can be made very small, so it can be used for particularly blood pressure detection during the exercise.

A metal strain gauge can be used as the strain gauge. This metal strain gauge may be a foil strain gauge. According to the present invention, an absolute value of a peak value of the pulse wave to be detected is unnecessary and not an absolute value of an arterial pressure but a change therein may be read. Therefore, it is possible to use a metal diaphragm type sensor including a metal strain gauge having a low gauge factor. The metal strain gauge is low in cost. Even when the sensor is placed in a position slightly displaced from the beat region, the strain can be detected by the entire metal diaphragm. The strain can be detected with the entire area that is relatively wide. Thus, even when a measurement region is displaced by the displacement of the sensor, a constant output can be obtained.

It is possible to obtain a strain sensor for blood pressure detection in which the strain gauge is sandwiched between two metal thin plates, namely, the strain gauge is sandwiched between the metal thin plate and another metal thin plate. When the strain gauge is sandwiched between the two metal thin plates, a thickness of the strain sensor can be significantly reduced. In a case where the strain sensor for blood pressure detection can be used for a blood pressure detecting device described later, when the strain sensor for blood pressure detection is interposed between a cuff and the living body, the strain sensor for blood pressure detection can receive a pressure from a surface of the living body to one sensor side and a pressure from a surface of the cuff to the other sensor side. Therefore, the pulse wave can be accurately detected while a cuff pressure gradually reduces.

Further, according to the present invention, there is provided a blood pressure detecting device, including: pulse wave detecting means for detecting a pulse wave, including a strain sensor for blood pressure detection that is provided therein; compression means for compressing a blood vessel of a living body and gradually reducing a compression pressure to the blood vessel; and output means for outputting the pulse wave obtained from the pulse wave detecting means while an occluded artery is gradually opened.

According to the present invention, there is provided a blood pressure detecting device including: pulse wave detecting means including a strain sensor for blood pressure detection that is provided therein; compression means for compressing a blood vessel of a living body and gradually reducing a compression pressure to the blood vessel; and blood pressure determining means for determining, as a maximum blood pressure, a pressure at a time when a first notch is caused in a waveform of the pulse wave obtained from the pulse wave detecting means while an occluded artery is gradually opened and determining, as a minimum blood pressure, a pressure at a time when the first notch is lost. Assume that, in this specification and claims, a term “notch” may indicate a valley part in which a portion of a peak of the pulse waveform becomes concave and is also called a negative spike.

According to the present invention, the pulse wave detecting means including the strain sensor for blood pressure detection that is provided therein can be used, so the pulse wave can be simply and directly detected from the living body to obtain the pulse waveform. A frequency characteristic of the detected pulse wave is a specific characteristic including the notch. Therefore, even when a band-pass filter or the like is used, the notch can be clearly distinguished from a noise. Thus, accurate maximum and minimum blood pressures can be detected based on the pulse wave.

The above-mentioned strain sensor for blood pressure detection can be used as the strain sensor for blood pressure detection that is included in the blood pressure detecting device. When the above-mentioned strain sensor for blood pressure detection is used, the beat of the blood vessel can be accurately converted into an electrical signal. Therefore, a blood pressure detecting device capable of detecting the accurate blood pressures is obtained.

According to the present invention, the blood pressure detecting device can include the compression means capable of compressing the blood vessel of the living body and gradually reducing the compression pressure to the blood vessel, so the occluded artery can be gradually opened. The compression means and the pulse wave detecting device can be combined to obtain the pulse waveform including the notch. The notch can be used to determine the maximum blood pressure and the minimum blood pressure.

The blood pressure detecting device can include the output means for outputting the pulse wave obtained from the pulse wave detecting means while the occluded artery is gradually opened. Because the output means for outputting the pulse wave obtained from the pulse wave detecting means while the occluded artery is gradually opened is included, the occurrence and absence of the notch can be recognized with reference to the outputted pulse waveform by the eyes of a person. Therefore, the maximum blood pressure and the minimum blood pressure can be visually determined.

The blood pressure detecting device can include the blood pressure determining means for determining, as the maximum blood pressure, the pressure at the time when the first notch is caused in the waveform of the pulse wave obtained from the pulse wave detecting means including the strain sensor for blood pressure detection while the occluded artery is gradually opened and determining, as the minimum blood pressure, the pressure at the time when the first notch is lost. Because the blood pressure determining means is included, when the pulse wave is detected from the artery that is gradually released from a compression state, the maximum blood pressure and the minimum blood pressure can be determined based on the pulse wave in which the notch is caused and lost.

As described above, the pulse wave obtained from the pulse wave detecting means including the strain sensor for blood pressure detection while the occluded artery is gradually opened can be used. Therefore, the maximum and minimum blood pressures can be determined without depending on the peak value of the pulse wave, it is unnecessary to detect the absolute value of the arterial pressure, and it is possible to use the strain sensor for blood pressure detection including no compression control means for controlling the compression pressure for compressing the living body. It is unnecessary to detect changes in Korotkoff sound and inner cuff vibration, so that the maximum and minimum blood pressures can be accurately and reliably obtained. Even when a heart beat is weak, a change in waveform is clear, so the maximum and minimum blood pressures of a severe patient having a weak heart beat can be detected. The maximum and minimum blood pressures are obtained based on the change in waveform, so the noise can be easily distinguished and the maximum and minimum blood pressures during the exercise can be detected.

According to the present invention, a sensor portion of the strain sensor for blood pressure detection can be attached to a part of a cuff that is the compression means. Because the sensor portion of the strain sensor for blood pressure detection can be attached to the part of the cuff that is the compression means, a pressure including a cuff pressure can be sensed by the strain sensor for blood pressure detection. Therefore, an electrical signal into which the pressure including the cuff pressure is converted can be processed to obtain a blood pressure value.

According to the present invention, a blood pressure detecting device for measurement during an exercise can include a separate band for integrally coupling the cuff that is the compression means to the strain sensor for blood pressure detection and for locating the sensor portion of the strain sensor for blood pressure detection at a distance from the cuff. Because the separate band for integrally coupling the cuff that is the compression means to the strain sensor for blood pressure detection and for locating the sensor portion of the strain sensor for blood pressure detection at the distance from the cuff is further included, the strain sensor is easy to handle particularly at the time of the exercise, that is, in a state in which the body moves. In addition, the strain sensor hardly picks up a noise from the cuff.

Further, according to the present invention, there is provided a blood pressure detecting method of detecting a maximum blood pressure and a minimum blood pressure based on a pulse wave propagating through an artery, including: using a strain sensor for blood pressure detection; determining, as the maximum blood pressure, a pressure at a time when a first notch is caused in pulse waveforms obtained while an occluded artery is gradually opened; and determining, as the minimum blood pressure, a pressure at a time when the first notch is lost.

According to a blood pressure detecting method of detecting a maximum blood pressure and a minimum blood pressure based on a pulse wave propagating through an artery, a strain sensor for blood pressure detection can be used. The pressure at the time when the first notch is caused in the pulse waveforms obtained while the occluded artery is gradually opened is determined as the maximum blood pressure. The pressure at the time when the first notch is lost is determined as the minimum blood pressure. Therefore, the maximum and minimum blood pressures can be accurately detected. Even when the beat is weak or the exercise is being performed, the maximum and minimum blood pressures can be accurately detected as in a normal state.

There is provided the blood pressure detecting method of detecting the maximum blood pressure and the minimum blood pressure based on the pulse wave propagating through the artery, further including a step of outputting, of the pulse waveforms obtained using the strain sensor for blood pressure detection while the occluded artery is gradually opened, a pulse waveform at the time when the first notch is caused and a pulse waveform at the time when the first notch is lost.

According to the blood pressure detecting method of detecting the maximum blood pressure and the minimum blood pressure based on the pulse wave propagating through the artery, of the pulse waveforms obtained using the strain sensor for blood pressure detection while the occluded artery is gradually opened, a pulse waveform at the time when the first notch is caused and a pulse waveform at the time when the first notch is lost are outputted. Therefore, the maximum blood pressure and the minimum blood pressure can be recognized with eyes or the like based on the outputted pulse waveforms.

Furthermore, according to the present invention, there is provided a blood pressure detecting program for obtaining a maximum blood pressure and a minimum blood pressure based on a pulse wave propagating through an artery, which is executed by a computer, including: a process for calculating, for each unit time, a change in pulse wave that is converted into an electrical signal by a strain sensor for blood pressure detection using a predetermined calculation expression; and a process for determining a time when a notch is caused in a waveform of the pulse wave and a time when the notch is lost based on the calculated change in pulse wave.

The process for calculating, for each unit time, the change in pulse wave that is converted into the electrical signal by the strain sensor for blood pressure detection is executed by the computer using the predetermined calculation expression. Therefore, the occurrence and absence of the notch can be detected as the change for each unit time. In addition, the process for determining the time when the notch is caused in the waveform of the pulse wave and the time when the notch is lost based on the calculated change in pulse wave is executed by the computer. Therefore, it is unnecessary to read by a change in pulse waveform by a person and routine processing can be performed, so that the maximum and minimum blood pressures can be accurately and simply obtained.

According to the strain sensor for blood pressure detection of the present invention, the beat of the living body can be accurately picked up, so that the strain sensor can be preferably applied to a blood pressure detecting device for detecting the blood pressures based on the pulse wave.

According to the blood pressure detecting device, the blood pressure detecting method, and the blood pressure detecting program of the present invention, the principle is clear unlike the conventional blood pressure measuring methods. Therefore, the maximum blood pressure and the minimum blood pressure can be accurately and reliably obtained.

According to the blood pressure detecting device, the blood pressure detecting method, and the blood pressure detecting program of the present invention, it is possible to obtain the maximum blood pressure and the minimum blood pressure of, for example, a severe patient having a weak cardiac output in an operation room or an ICU.

According to the blood pressure detecting device, the blood pressure detecting method, and the blood pressure detecting program of the present invention, the maximum blood pressure and the minimum blood pressure can be obtained under an exercise stress such as a cardiovascular exercise stress test.

The above description of the present invention should not be construed restrictively; the advantages, features, and uses of the present invention will become still more apparent from the following description given with reference to the accompanying drawings. Further, it should be understood that all appropriate modifications made without departing from the gist of the present invention are covered by the scope of the present invention.

### BRIEF DESCRIPTION OF THE DRAWINGS

Throughout the drawing figures, like reference numerals will be understood to refer to like parts and components. In the accompanying drawings:

FIGS. 1A and 1B show an end portion of a blood pressure detecting device according to an embodiment of the present invention, in which FIG. 1A is a sectional view obtained along the SA-SA line shown in FIG. 1B and FIG. 1B is a plan view thereof;

FIG. 2 is a perspective view showing a state in which the blood pressure detecting device is attached to an upper arm of a living body;

FIGS. 3A and 3B show a strain sensor for blood pressure detection, in which FIG. 3A is a front view showing a pressure transducer thereof and FIG. 3B is a plan view showing the strain sensor for blood pressure detection;

FIG. 4 is a block diagram showing circuits for processing data obtained by the strain sensor for blood pressure detection;

FIGS. 5A and 5B show an end portion of a blood pressure detecting device according to another embodiment of the present invention, in which FIG. 5A is a sectional view obtained along the SB-SB line shown in FIG. 5B, FIG. 5B is a plan view thereof and FIG. 5C is a front view showing a pressure transducer thereof;

FIG. 6 is a time chart showing a waveform of a pulse wave detected by the strain sensor for blood pressure detection;

FIG. 7 is a block diagram showing a blood pressure detecting device according to an embodiment of the present invention;

FIG. 8 is a block diagram showing a blood pressure detecting device according to another embodiment of the present invention; and

FIG. 9 shows a relationship between frequency-decomposed Korotkoff sounds and maximum and minimum blood pressures.

### DETAILED DESCRIPTION OF THE PREFERRED EMBODIMENTS

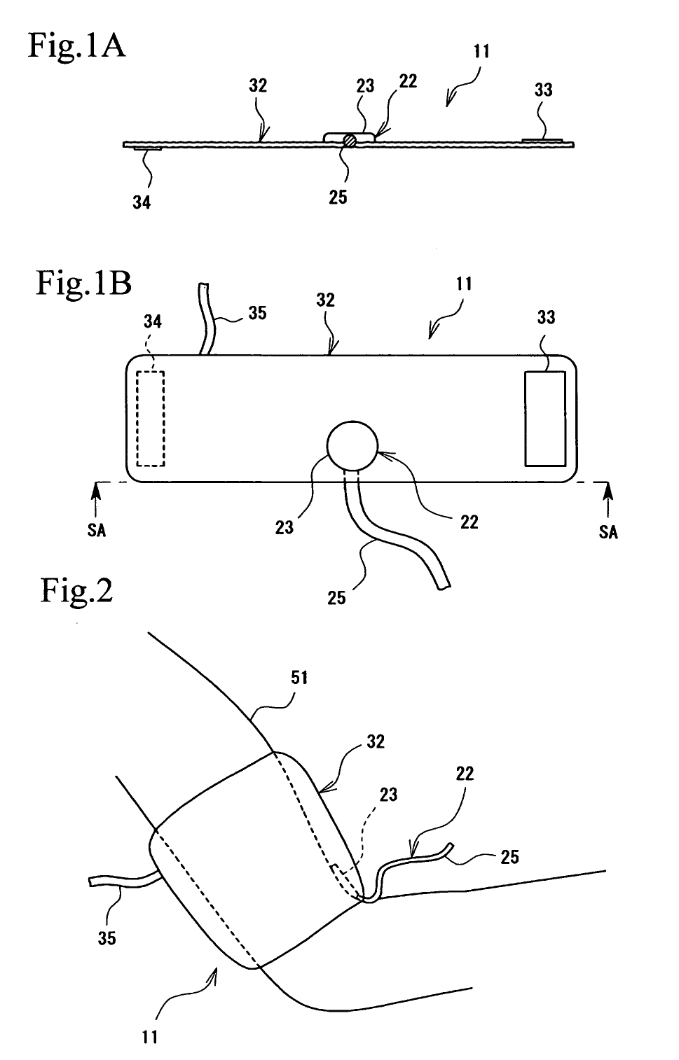

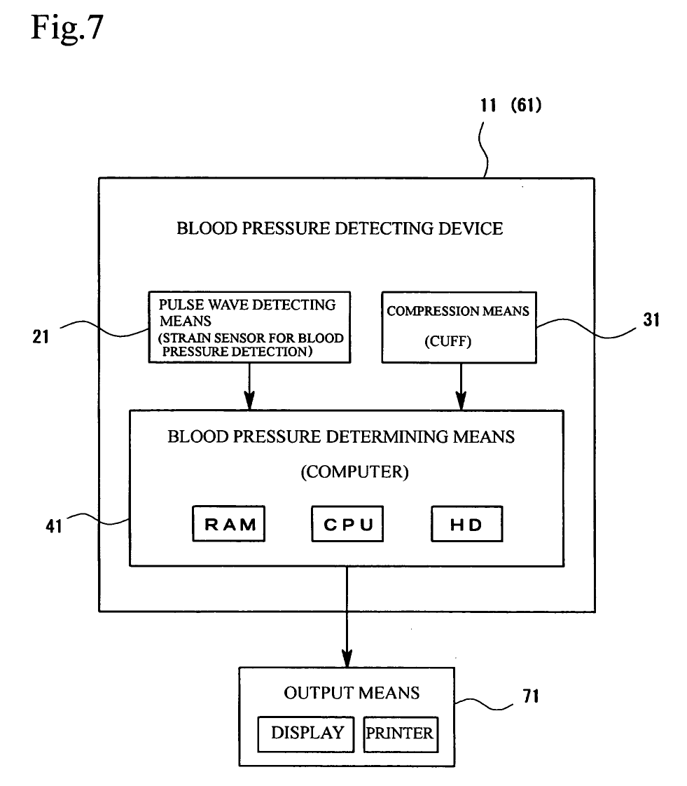

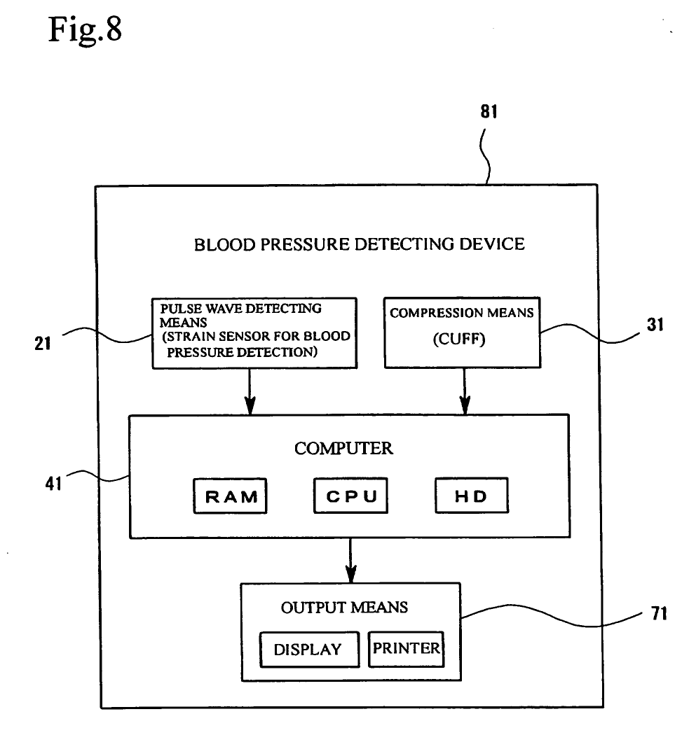

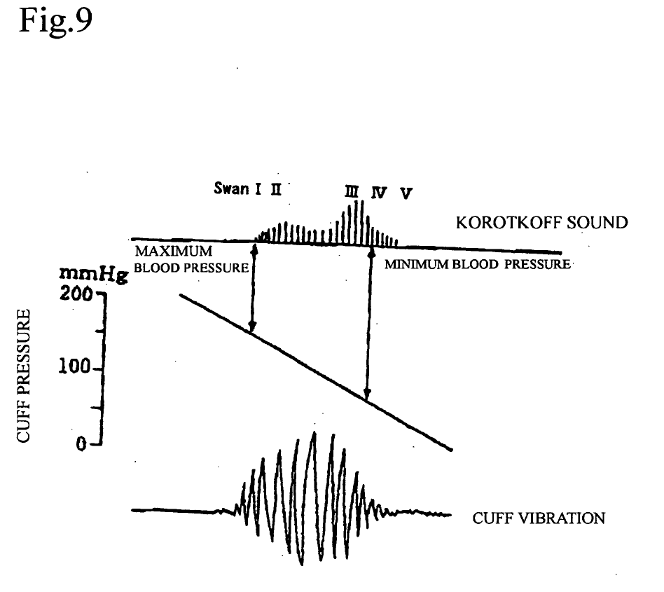

Hereinafter, the present invention will be described in detail with reference to embodiments thereof. As shown in FIGS. 7 and 8, each of blood pressure detecting devices 11, 61, and 81 according to the embodiments may include pulse wave detecting means 21 and compression means 31. Each of the blood pressure detecting devices 11, 61, and 81 may further include blood pressure determining means 41 or output means 71. A first embodiment of the present invention will be described. FIGS. 1A and 1B show a measurement end portion of the blood pressure detecting device 11. As shown in FIGS. 1A and 1B, a strain sensor 22 for blood pressure detection, serving as the pulse wave detecting means 21 is provided on a center end portion of a cuff 32 serving as the compression means 31. As shown in FIG. 2, the blood pressure detecting device 11 can be used with a state in which, for example, the cuff 32 is wound around an upper arm 51 so as to locate the strain sensor 22 for blood pressure detection above a brachial beat region.

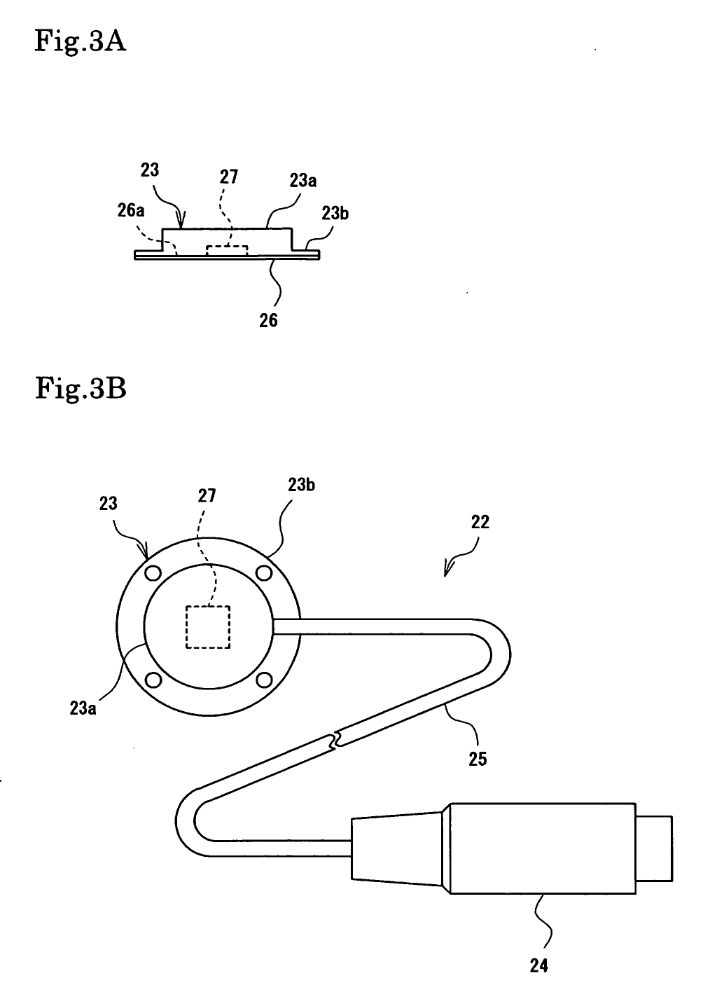

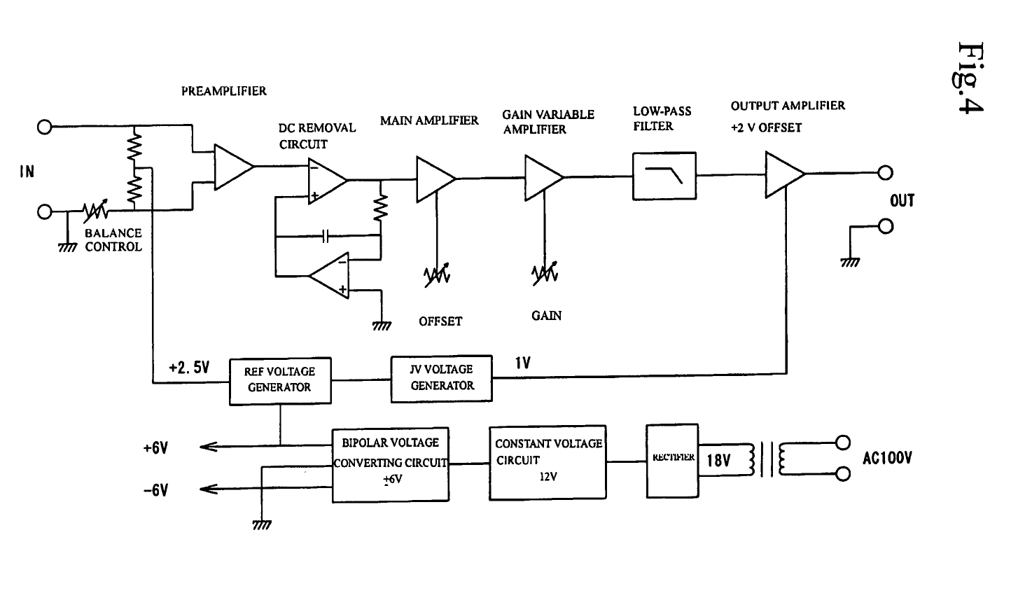

The pulse wave detecting means 21 can be used to detect a pulse wave mainly from a beat region of a living body. The strain sensor 22 for blood pressure detection may directly detect a pulse wave from a measurement region. FIGS. 3A and 3B are external views showing the strain sensor 22 for blood pressure detection. As shown in FIG. 3A, a pressure transducer 23 has a hat shape whose diameter is approximately 30 mm and thickness is approximately 5 mm to 20 mm. The pressure transducer 23 is coupled to a mini DIN plug (4P) 24 connected with an amplifier (not shown) through a code 25. The pressure transducer 23 has a strain gauge 27. The strain gauge 27 may be a metal strain gauge 27. The strain gauge 27 may include a semiconductor strain gauge or a foil strain gauge. The strain gauge 27 can be provided on a rear surface 26a of a metal thin plate 26. The metal thin plate 26 can be a phosphor bronze plate. The metal thin plate 26 is exposed in a surface of the pressure transducer 23. When the pressure transducer 23 receives a pressure (artery pressure) of the living body, a resistance of the strain gauge 27 changes. Based on this fact, the pressure is converted into an electrical signal. The electrical signal is amplified by an amplifier (not shown) and a noise of the signal is removed (FIG. 4) to detect a pulse waveform.

Shown as a hat shape within FIGS. 3A and 3B, the pressure transducer 23 has a crown portion 23a and a brim portion 23b. In the case of the metal thin plate 26 having a circular shape shown in FIGS. 3A and 3B, a diameter of the metal thin plate 26 is 5 mm to 30 mm, preferably, 5 mm to 20 mm. But when the metal thin plate 26 is formed in a rectangular shape, an average length of each of the long side and the short side is 5 mm to 30 mm, preferably, 5 mm to 20 mm. When the diameter of a circular metal thin plate 26 is shorter than 5 mm or when a side of a rectangular metal thin plate 26 is shorter than 5 mm, it is difficult to place the strain sensor 22 for blood pressure detection immediately above the beat region. When the diameter of a circular metal thin plate 26 exceeds 30 mm or when a side of a rectangular metal thin plate 26 exceeds 30 mm, an increase in noise captured by the pressure transducer 23 results, so that it is unlikely to accurately transfer a pressure to the pressure transducer 23. Therefore, when the diameter of a circular metal thin plate 26 is shorter than 5 mm or longer than 30 mm or when a side of a rectangular metal thin plate 26 is shorter than 5 mm or longer than 30 mm, the pressure of the blood vessel cannot be accurately detected. The reason why 5 mm to 20 mm is preferable is that the pressure applied to the metal thin plate 26 can be transferred to the pressure transducer 23 without any change and is hardly affected by the noise. The thickness of the metal thin plate 26 is 2 mm or less, which is a thickness capable of maintaining a shape that acts as the metal thin plate 26. This is because, when the thickness of the metal thin plate 26 exceeds 2 mm, the pressure of the blood vessel that is to be transferred to the strain gauge 27 is reduced. The minimum thickness of the metal thin plate 26 that capable of maintaining a shape is approximately 0.1 mm. This thickness of the metal thin plate 26 described above may depend upon the kind of metal used as the metal thin plate 26.

The metal thin plate 26 may be made of metal in which an elastic coefficient is low, a property is flexible, and a strength is high. Specifically, the metal thin plate 26 can be a copper array. As compared with a hard material such as a stainless steel, for example, phosphor bronze, brass, or bronze has a Young's modulus of 130 GPa or less and a shearing modulus of 4.5 GPa or less, which the elastic coefficient is low and it is easy to bend. Therefore, the pressure of the living body is easily transferred to the pressure transducer 23 without any reduction. Of phosphor bronze, brass, and bronze, the phosphor bronze has a high Poisson's ratio and instantaneously returns to an original shape, so that the phosphor bronze is a more preferable as a material for the metal thin plate 26 to accurately reflecting the beat of the blood vessel.

Unlike a pressure sensor used for the conventional tonometry method, it is unnecessary that the strain sensor 22 for blood pressure detection may include pressure applying means for pressing metal thin plate 26 serving as a diaphragm from an inner sensor portion. Therefore, pressure control means for controlling a pressure of the inner sensor portion is also unnecessary.

The number of strain gauges 27 provided on the metal thin plate 26 is not particularly limited. For example, a single strain gauge 27 can be provided on the metal thin plate 26. Likewise, more than one strain gauge 27 being provided on the metal thin plate 26 is also within the scope of the invention. It is only necessary to provide one to several strain gauges 27. When the number of strain gauges 27 increases, changes can be picked up at various positions on the metal thin plate 26. Unlike the tonometry method, it is unnecessary to detect the pressure at the position immediately above the artery. In addition, it is unnecessary to obtain an absolute value of an arterial pressure and it is only necessary to pick up a waveform in which the pressure changes. Therefore, strain may be detected at any region on the entire metal thin plate 26. However, in order to obtain a more accurate waveform, when one strain gauge 27 is used, the strain gauge 27 is preferably located at the center of the metal thin plate 26. When a plurality of strain gauges 27 are used, the strain gauges 27 are preferably located on the circumference of a concentric circle at regular intervals. A semiconductor strain gauge having a gauge factor of −80 to −150 or a high gauge factor of approximately 60 to 300 can be used as the strain gauge 27. It is also possible to use a metal strain gauge as the strain gauge 27. The gauge factor for the strain gauge 27 may be approximately 1.5 to 10 or may be a low gauge factor of 2 or less. When the semiconductor strain gauge is used as the strain gauge 27, a size of a sensor chip becomes smaller, so that the sensor chip can be made more compact. When the diameter or the side of the metal thin plate 26 is large, an area of the metal thin plate 26 is large, so a metal strain gauge such as a foil strain gauge, having a long base length of approximately 10 mm to 25 mm can be also incorporated in the sensor chip. When the foil strain gauge is used, the sensor chip can be manufactured with lower cost. The pressure detected by the pressure transducer 23 is converted into an electrical signal. That is, a change in resistance that is caused by the strain of the strain sensor 22 for blood pressure detection is converted into a change in voltage by a Wheatstone bridge circuit or the like. Then, the voltage is amplified by an amplifier, for example, the circuit shown in FIG. 4. A noise and a cuff pressure signal are removed from the amplified voltage to generate a resultant signal as the pulse wave.

A pressure applying pump (not shown) for introducing air into the cuff 32 and a compression band such as the cuff 32 as shown in FIGS. 1A, 1B, and 2 can be used for the compression means 31 provided to occlude the artery. The cuff 32 has a structure in which a pouched rubber tube is enclosed with a cloth. An outer shape of the cuff 32 is a flat rectangle. The cuff 32 may include Velcro (registered trademark) fasteners 33 and 34 stitched in both ends thereof and can be held thereby with a state in which the cuff 32 wound around the upper arm 51 of the living body. A pressure can be applied from the pressure applying pump to the cuff 32 through a pipe 35, so that the rubber tube can be expanded to compress the upper arm 51 around which the cuff 32 is wound. The air can be exhausted from the cuff 32 to an outside through the pipe 35. A pressure sensor for sensing the inner pressure of the cuff 32 is coupled to the cuff 32 through the pipe 35, so that the cuff pressure can be controlled.

The pressure transducer 23 serving as a sensor section is provided in a center portion of the band-shaped cuff 32 in a long-side direction and an end portion thereof in a short-side direction. Therefore, as shown in FIG. 2, when the cuff 32 is wound around the upper arm 51 and the cuff 32 is held by, for example, the Velcro (registered trademark) fasteners 33 and 34 stitched therein, the strain sensor 22 for blood pressure detection can be pressed and held at a low pressure of approximately 10 mmHg with a state in which the pressure transducer 23 is located immediately above a brachial artery beat region. The strain sensor 22 for blood pressure detection included in the blood pressure detecting device 11 can accurately detect the pulse waveform even when a slight variation in position occurs.

A plate cover can be provided on the metal thin plate 26 in which the pressure transducer 23 is in contact with the living body. A plate cover for the metal thin plate 26 may include, for example, a cover produced using an aqueous resin solution. When this solution is applied onto the metal thin plate 26 to form a coating, a cool feeling of the metal thin plate 26 can be avoided to provide a warm feeling to the living body. When the plate cover is replaced for each person to be examined, the metal thin plate 26 can be maintained in a clean and sensitive state.

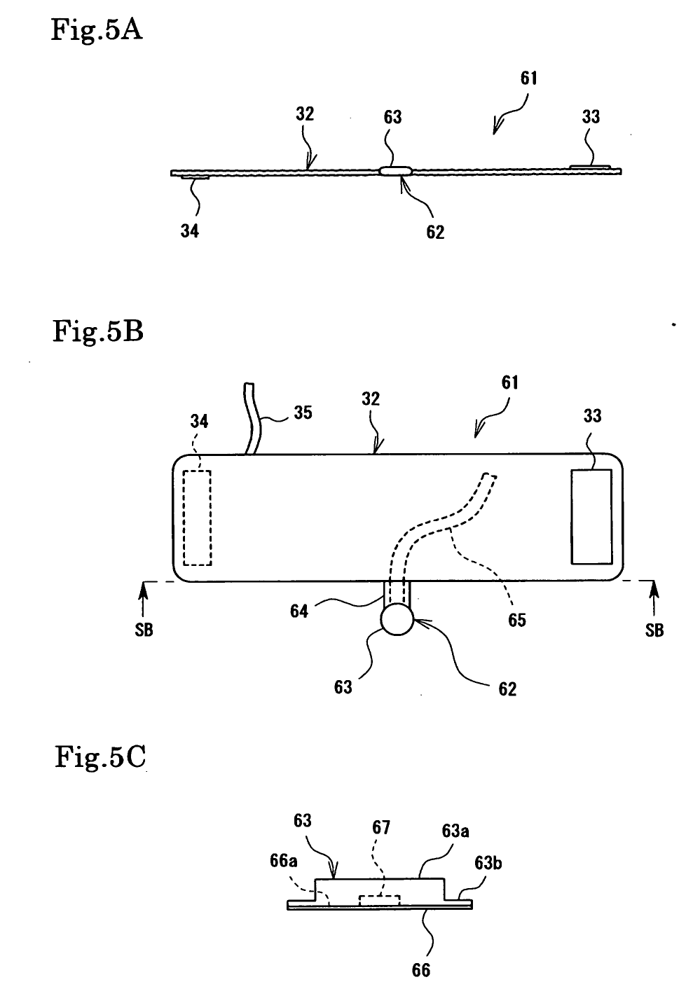

The blood pressure detecting device 61 according to another embodiment of the present invention as shown in FIGS. 5A and 5B can be used in a cardiovascular exercise stress test or the like. A pressure transducer 63 serving as a sensor section of a strain sensor 62 for blood pressure detection may be separated from the blood pressure detecting device 61 by a distance corresponding to a length of a band 64 coupled to the cuff 32. The pressure transducer 63 has a diameter of 20 mm or less, preferably, a diameter of 5 mm to 7 mm and a thickness of 1 mm. As shown in FIG. 5C, the pressure transducer 63 has a strain gauge 67 like the pressure transducer 23. The strain gauge 67 may be a metal strain gauge 67. The strain gauge 67 may include a semiconductor strain gauge or a foil strain gauge. The strain gauge 67 can be provided on a rear surface 66a of a metal thin plate 66. The metal thin plate 66 can be a phosphor bronze plate. The metal thin plate 66 is exposed in a surface of the pressure transducer 63. The pressure transducer 63 is also connected with an amplifier (not shown) through a code 65. The band 64 can be used to hold the pressure transducer 63 in the cuff 32. While the band 64 can be made of a cloth, the band 64 is not limited to cloth or to any specific material. In addition to the cuff 32 wound around the upper arm at the time of blood pressure detection for holding the pressure transducer 63 to a beat region, the pressure transducer 63 can also be held to the beat region by a rubber band, an adhesive tape, or the like.

The pressure transducer 63 is integrally provided with the cuff 32 at a distance from the cuff 32 through the band 64 serving as a separate band. Therefore, the influence of an external pressure applied to the cuff 32 during an exercise on the pressure transducer 63 can be prevented.

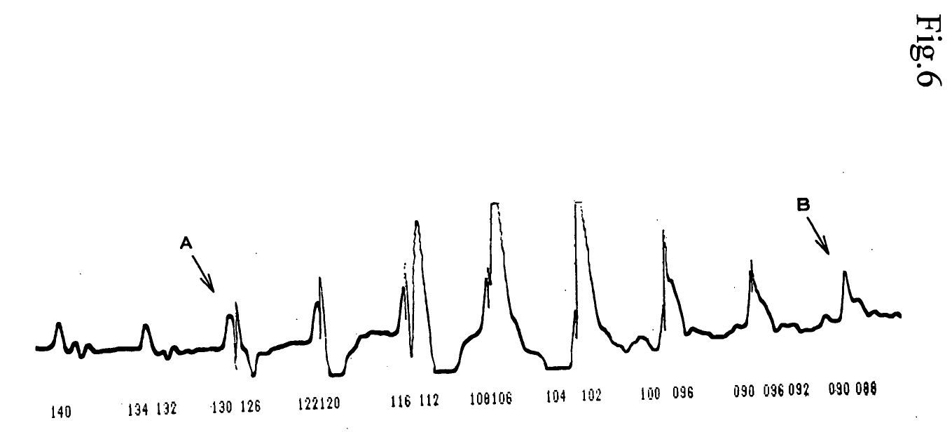

The blood pressure determining means 41 may determine the maximum blood pressure and the minimum blood pressure based on a feature of the obtained pulse waveform. The maximum blood pressure and the minimum blood pressure are obtained based on a change in pulse waveform after the occluded artery is opened. FIG. 6 shows a pulse waveform produced while the upper arm is compressed at the cuff pressure that exceeds the maximum blood pressure to block the blood flow and then the cuff pressure is gradually reduced at a predetermined rate. This pulse waveform is outputted by the output means 71. In the pulse waveform, a blood pressure at a time when a negative notch that is not included in a preceding pulse waveform is recognized as a forward waveform component (“A” shown in FIG. 6) is determined as the maximum blood pressure. In addition, a blood pressure at a time when the negative notch is lost (“B” shown in FIG. 6) is determined as the minimum blood pressure. As described above, the blood pressure determining means 41 may identify the maximum blood pressure and the minimum blood pressure corresponding to the occurrence and absence of the negative notch of the pulse waveform. The maximum blood pressure and the minimum blood pressure obtained using this method are equal to a maximum blood pressure and a minimum blood pressure measured using an invasive method of inserting a catheter into a radial artery, and thus these blood pressures are accurate values.

The reason why the maximum blood pressure and the minimum blood pressure can be detected corresponding to the occurrence and absence of the notch may be as follows. While the cuff pressure is gradually reduced after the upper arm is compressed at the cuff pressure to occlude the artery, the slight blood flow from the artery that is being occluded at the cuff pressure starts again at the maximum blood pressure. Therefore, the negative notch is caused in the pulse waveform. On the other hand, the artery that is being occluded at the cuff pressure is fully opened at the minimum blood pressure, so that the notch is completely lost.

Because the maximum blood pressure and the minimum blood pressure are detected corresponding to the occurrence and absence of the notch in the pulse waveform, it is unnecessary to obtain a maximum point and a minimum point of the pulse wave and an absolute value of the pressure of the artery. Therefore, it is only necessary to detect a change in waveform, so that the strain sensor 22 for blood pressure detection in which the artery pressure is applied to the entire metal thin plate 26 and the strain sensor 62 for blood pressure detection in which the artery pressure is applied to the entire metal thin plate 66 are preferably used.

FIG. 7 is a block diagram showing the blood pressure detecting device 11. The blood pressure detecting device 11 may include the pulse wave detecting means 21, the compression means 31, and the blood pressure determining means 41. The blood pressure determining means 41 may include a computer and a computer program for starting the computer. The computer has an arithmetic processor such as a central processing unit (CPU), a random access memory (RAM), and a hard disc (HD) drive. For example, assume that a maximum and minimum blood pressure determining program recorded in an external recording medium such as a CD-ROM is read out on the RAM and executed by the CPU. In this case, a change in current value per unit time is detected based on, for example, a differential value of the pulse wave data obtained by the pulse wave detecting means 21. The occurrence and absence of the notch are identified based on the change in current value to determine the maximum blood pressure and the minimum blood pressure. Then, the obtained maximum blood pressure and the minimum blood pressure which are to be used are displayed on a display of the output means 71 or printed by a printer thereof together with, for example, data including a patient name, sex, and age.

FIG. 8 is a block diagram showing the blood pressure detecting device 81 according to another embodiment of the present invention. The blood pressure detecting device 81 may include the pulse wave detecting means 21, the compression means 31, and the output means 71. The blood pressure detecting device 81 can operate as in the blood pressure detecting device 11 shown in FIG. 7. The data obtained from the pulse wave detecting means 21 and the compression means 31 are processed by the computer and then displayed as the pulse wave on the output means 71. The maximum blood pressure and the minimum blood pressure can be determined by visually recognizing the pulse wave outputted to the output means 71 by a person.

Each of the above-mentioned embodiments is merely an example of the present invention and thus modifications can be made without departing from the spirit of the present invention. For example, the strain sensor 22 for blood pressure detection may be an elastic diaphragm type using a plastic material or the like, other than a semiconductor diaphragm type and a metal diaphragm type. The blood pressure determining means 41 may be means for outputting the pulse wave obtained from the pulse wave detecting means 21 without any processing in order to read the maximum blood pressure and the minimum blood pressure from the pulse wave by the eyes of a person. Although the maximum and minimum blood pressures are detected corresponding to the occurrence and absence of the notch in the pulse waveform, the maximum and minimum blood pressures may be detected based on a change in electrical signal that exhibits the occurrence and absence of the notch. The improvement can be made so as to perform the data transfer from the strain sensor for blood pressure detection to the blood pressure determining means in a cordless state.

1. A strain sensor for blood pressure detection, comprising:
a pressure transducer including:
a metal thin plate for receiving a beat of a living body; and
a strain gauge provided on a surface of the metal thin plate, for detecting a pressure based on the beat propagating through the metal thin plate.

2. A strain sensor for blood pressure detection according to claim 1, wherein the strain gauge comprises a semiconductor strain gauge.

3. A strain sensor for blood pressure detection according to claim 1, wherein the strain gauge comprises a foil strain gauge.

4. A strain sensor for blood pressure detection according to claim 1, wherein the strain gauge is sandwiched between the metal thin plate and another metal thin plate.

5. A strain sensor for blood pressure detection according to claim 1, wherein the metal thin plate has a diameter of 5 mm to 20 mm and a thickness that is equal to or smaller than 2 mm and corresponds to a thickness for maintaining a thin plate shape and the metal thin plate comprises a copper alloy.

6. A strain sensor for blood pressure detection according to claim 5, wherein the metal thin plate comprises a phosphor bronze plate.

7. A blood pressure detecting device, comprising:
pulse wave detecting means for detecting a pulse wave, including a strain sensor for blood pressure detection that is provided therein; and
compression means for compressing a blood vessel of a living body and gradually reducing a compression pressure to the blood vessel.

8. A blood pressure detecting device according to claim 7, further comprising output means for outputting the pulse wave obtained from the pulse wave detecting means while an occluded artery is gradually opened.

9. A blood pressure detecting device according to claim 7, further comprising blood pressure determining means for determining, as a maximum blood pressure, a pressure at a time when a first notch is caused in a waveform of the pulse wave obtained from the pulse wave detecting means while an occluded artery is gradually opened and determining, as a minimum blood pressure, a pressure at a time when the first notch is lost.

10. A blood pressure detecting device according to claim 7, wherein the strain sensor for blood pressure detection comprises:
a pressure transducer including:
a metal thin plate for receiving a beat of a living body; and
a strain gauge provided on a surface of the metal thin plate, for detecting a pressure based on the beat propagating through the metal thin plate.

11. A blood pressure detecting device according to claim 7, wherein:
the compression means comprises a cuff; and
the strain sensor for blood pressure detection comprises a sensor portion attached to a part of the cuff that is the compression means.

12. A blood pressure detecting device according to claim 7, further comprising a separate band for integrally coupling the cuff that is the compression means to the strain sensor for blood pressure detection and locating the sensor portion of the strain sensor for blood pressure detection at a distance from the cuff to allow measurement during an exercise.

13. A blood pressure detecting method of detecting a maximum blood pressure and a minimum blood pressure based on a pulse wave propagating through an artery, comprising:
using a strain sensor for blood pressure detection;
determining, as the maximum blood pressure, a pressure at a time when a first notch is caused in pulse waveforms obtained while an occluded artery is gradually opened; and
determining, as the minimum blood pressure, a pressure at a time when the first notch is lost.

14. A blood pressure detecting method according to claim 13, further comprising outputting, of the pulse waveforms, a pulse waveform at the time when the first notch is caused and a pulse waveform at the time when the first notch is lost.

15. A blood pressure detecting program for obtaining a maximum blood pressure and a minimum blood pressure based on a pulse wave propagating through an artery, which is executed by a computer, comprising:
a process for calculating, for each unit time, a change in pulse wave that is converted into an electrical signal by a strain sensor for blood pressure detection using a predetermined calculation expression; and
a process for determining a time when a notch is caused in a waveform of the pulse wave and a time when the notch is lost based on the calculated change in pulse wave.
